SEQUENCE LISTING (4 sequences)

SEQ ID NO: 1 - DNA, 11839 bp, Artificial Sequence
  Active LTR retrotransposon
tgtgagaacccgtaaaaatcccttaataattttcccaggggtttttatttcctttgagaacccgttaaaaaccctaataatttttcccgagggtttatattcccctttaatagcatgttttctgcatttcctggcttaggaattttcctgaggaattttatgagtaattatagttttagtatgatttttcttagccttggagagtttttggtaaaattaagagtatataatggacgtgggacccacaagtgcgaaaagttcggaaaaaaattcggccaattaggttaagtttcggatactgtgtaaaatttatcgggtgtttaagagataagtagagtgtgtgaagtgattgatgtgagaggaaaaaaaaagaaaaaaaagataagaatgcattaatgagtgtgacaagtgtcactctctgattggtttttactttaagattttactattccaattttttgacttttttgaacaaaggttaataaatatcttaaaaattgcccaaaaatcaccatttcttacctttctggatggccgggccctctctagcaagaagaaagacaaaaactcttcaacatcttggcaaattccctagctcaaatcatccaaactacttgcctaaatttagtttttactccataaaatttccttccttggggtgctagtaggtagttttagtgaagtttgtttgaagagctaaaggtgtccaacatctctatctctcttggttactaggtgagtggtgtgtgaatttcccctcttgcactttaatgaagcttaagttatgcttattagtgattaaagtggctgaaatttctggttttcacatcttggtttgaagtgatttgttggtgtaagtttttattttattgatgatttttctggtttaattggattatgatggttgtggttgtttaatgatgattgatacatatgaggggttatgactctagtggatggaaatgattgataatttgcaaccaattttgggatttgggaattaaattttgggaaagttagggttcatgggaagctacattctgcccgaatttattgcaccatgttagaggccgaattggccaacttggcttaaaaacatgaaaagttgtaggtattgatgtgtttgaagtgcctgcaaaaatttcaggtcatttggattagtgtagagtgagatatgtcgattttactgtttgctgttctgggttgatcagaatgcgaaaactgcacttgtaattggccattttttgactggaattgcttttggattttggatgttggtgtaacttctgatgaaatgtagcttgaatgtcttagctaccatatgccttttggaatcaatgcatttggacctgtatttagacttgagttgtactaattacagcattgtgttgatttgttaaacctgcaattaacggttctggttttggtattctgcatttttggacactagttgtgctacggatttggaactgagtggccttcgtacatttgttgtagccctgtcttttagcttacgagaatggttcgagatggtgggtccttacaccctcatccgcataagtcgtagcgcattttgtgcccattacccgcattaggaggccaaaaactgttttttgtgtttgggccaaaatgagttacattttcctgattttctggtttgctataatgctggtatatgtttgcaaaaccctatttgggggttgtgattggcattgctttatgactcgttatcgagtctcattgtactttgcttacgtgttcgtagggcgtgacggtggttcacgacattctcctgacggagaagtgcatgaaatagcacttaattgattggtgagtatactactcacttaattaatgttacaatgtggcttttggtatatgtgaatttgatgccttgaaggctaattatggctattgaaaatagattgcggttggagggtggtacttgaccgccctcaccccccttcgtgattccattcatgactgttttaccgttttacttgaaatactgcacttggctatatgagatattgaattccattcatgaaaaactagatttggtgtcatttgggacgaatgtccaacggccttactgtattactgagctcaatcccgtaggtaggttgtcaattgaatcgagccggcgagggttttggtcgtgaaaaattgacatgccacgggggactgtactggggaaccttgtagtaatgagactcttgattccggtaaacctcgagtattaccaaaagtgactgaatggagtgcgggcccggttggggtatggtttggtggaaggaatggaagtgaagtgaggtgctacgggttgggtattcttaaacaattgacggagggtcaatgaggttggatcaagaatgtaagcgtggaaatgggattgggctctttgagagccgtccgtatccttttaccgacttgtttaattctcactccttaattgtgtacttgaaatgaaaggttatgctaaaggcaccttgttgctcttatatgctcttcgatgcttatgtggtagtaactcattgagcttaacctcacttccgttccatttgttttccttacaggaaaaaatgactattttgggaaaggttgtggctagttggttgtcaagttgagctcatgtaaatgtatattttgaatagctccgtgcttgagggtgaaaaccctaagtgttttgtttccaaccaatggttggtttgtaatttggctaattgcacatgtatgaacttgttggatattttggtatgccgctttgggcttgtaaatgttaaatttcgatgtttatatatgttggattgatgtttggttgaaactccggattctgacgtggccggcactgttcatcatcggcttcgattttcaattttttttaaatttatgttgtgattttgacttgtttgcgtcgcgttggtgtgttccggaaacgtagtagatcgacgtaaactgatccgttagtcctggcgagagctgggcagggcagtccgctaacccctttggttcgccttaggggaaggtggggctgttacaacaagggtggtatcagagcctaggtttgaattagcctggggtgttataggaatgcttgatctgagggtttattgtttatggtttttaagcttattgattcttatttatttacacttttggtgtaggatggacagttccagtggacctagtgatggccctgcggccaagcgaccccgtgtagcgctcccgatgattaagtaccgagagatccggccaaaagggggccgttattcgttggagcccgtctaaccgtcttcggcggtgcgcgtgtaaggagaagtatgcttacccgaatgccctcgttttggcagttgtgaaggaaagaaaaggagctggcaaaaagctaatgccaagcttgaggttgaggtgaatcagttaaaggcggccaacgaggggcaagccaaaaggatagaggatctgaagtacgatgtactagaggcggaggatagagtcgatgacctctgtgcccagctgagggaggcgcgtgagcgcgagtctaagagggctcgaaaggtgaagaatcgagccgctgcgatcctgaacctttgcgccgaggtggtcgagggcgtgagcgaaacgaggactcctgtgcggggtctgaggctggggttggatccactccgtcgggtgggtctcatagctcgtcgtcggtctaggacaggacttctaggtgtttgttgcgttttggatagttttggtcttgtatataagggcggatagggagtagggccttagctaatcattttgtacggctagattagctacatgtatatttcttttcaatgaggccattcccttggggattgtccatgctttgtacttgacttgactgaacttgacttgactgattgtatatatgtgtgttgtttgtcttgtttgtaatgtatatatgtgtgtttggaaagttttgagattttacttacatgcattgttattatattggtttagtaccattgcctagatcatataaagtttatggaaggaacacgtagtggacggggacgtgggcgcggaaatagacaacccacaccggataggggtattggggaaacctctactggacctaaccctgacccccaatgtgcaaatcgctgccgctatgcagcaaatgactaacttagctagcacaagtggtgcagcaacaaggccagaatacaaaccctggaaatcctggaaaaccctggtaaccacatcgagagcgaagacagagctctcgaacgttttcaaaagttttgctccaccccaaattcattgggggacctgatccggatgttgccgagcgatggctcgagaagatggttgatatttttgcggccctgcactacaccgaagaacggcaaggtgacttttgccgtattccagttcgaaggggcggcccgttcctggtggaacgtcatacgacaaaaatgggaacgggaacaaacgcccaggacttgggtgaatttcatccgggagtttaacgcgaaaattcttccctcctctggttcaggaaaggaaggaagatgagttcatccgactccgccaaggagctcaatcggtggcggaatacgagagccagttcacccgcctgtccaaatttgcgcctgagttgattgtgacggagcaacggcgaattaggcgtttttatccagggcctaaaacgtggagattcagaaggacctcgcagtggctcaaatcaatgcttttagcgaggccgttgagaaggcccaacgggtagagagtgccagattgcaagtacggaacttccaggcgaagaaagcggggttttcctggaagcagctctggacaaggggataaaggtaccccctccaagttcggacgacggagctggaggtggacgacagtcgggttccggcagaggggctcagtccaggggtggctcagccgggagaggccaaggcggaaacttccagaagggttccggttcggcatcccgtgggccttgtggatactgcggaaagccgaatcactccgaagatagttgctggaagaaagggggcaaatgtctgcgttgtggaagcgcagaccaccagcttgctacctgcccacttttaaagcaagacggaaaggggagctcaaaccgccgccaaagaccaatgctggaccagttaggccagctaaggggagatggggtccaaaacctaaggtgccagctcgagtgtattccttagaacctcaacaggtcccagattcctcagaggtcgtagaaggtacgatccctattttccaccgttttagccaaaggatttttaattgatcctggtcgccactcattcctttgttaaccctgatttcatgtgtggcatcgatataaaaacctgctagtttaccatacgacctagaagttagtacacctacggggaatcaacgtttggttactagtatggtttatagggattgtgaagtgtgggtaggagagaagagattgttaggggacttgataagtttggtcatttaaggggtatgatgtgatctttgggtatggactggctagccaagtataatgcccaactggattgtaaaacgaaagtggtagaattccgcattccctggtgaggcaaccttaaggttggacataaggggtaggttagcctcatctgctttgatttcgggcattcgggctagaaaactgctaagtagaggggcgcaaggatttttagcctttttgattaacacccctacggataagttaaaggtggaggacgtggccatagtaaaggaatttccggatgtatttacctgatgagttagtagccctacctccggaaagagagattgaattcaaaatagacctgctaccgggatcctcacctatctcaaaaacaccataccgaatggctcctgctgagctcaaggagctaaagttgcaattacaggatcttttggagcggggattcattcaagagagtggggtctccttgggggagctcctgtactgtttgtgaagaaaaaggacggtgggttgaggatgtgtatcgaattatcggggcctgaataatattacggtgaaaaataagtatccactgccccacatagatgagttgtttgatcagttgcagggagcggtggtcttttcgaaaactggatctccgccaagggtattaccagttattaatcaggaaggaggatattccgaaaaccgccttcaactcgagatatgggcatttacgagttcgtcagttatgccctttggactaaccaatgctcctgtccgccttcatggatttaatgcatagggtttttaagccctacctggatcgattttgtagtagtcttcatcgacgacattctggtctattccaagacacgtgaagagcatgagcagcacttgaggattgtcttacaaaccctaagagaccatcaactatatgccaagttcagcaagtgcgaattttggctggagaaaattgcttttctggggcacgtaatttctcatgagggaatttccgttgacccggcgaaagtagaggctgtgactaattggaagaggcccgaaagtcccacagaggttcgcagctttctaggactggctggatattatcggcgtttcatcaaggacttttctaaaactggccggtcctctaaccgacttctgacaaagaaacatggtcggttcatttggaatgctcgaagtgagacaagttttcaagagttaaagaagagattgaccatggctccagttctagtcttacctaacggaggtgacgggtttgctgtgtatacggacgcgtcacgagaaggcctgggttgtgtattaatgcagaaccgaaatgtgatctcttttgcctcaaaggaagttaaaacctcacgagcagaattatccaacccatgatctggagctagctgccgttgttttcgctcttaaaaagtggagacattaccttatatgggggtaaccttcgaggtttactccgatcacaaaagcctgaggtatctcttcttcacagaaggaattaaacatgaggcagcgacggtggatggaatttcttggaggattacgactgtaccatcaactaccatccagggaaaggcgaacgtagtggccgatgcccctaagtcgcaaagctcaggtcgcaagcttaatgatttaaagagtggaatttgctcgagtcggtttgtgagtggaaaccctgccttgggagccataaggtgaaaaggtgaatttttggcaatattaaagtgtcacacttccaccctcttggagagaattaaagaagctcaaaaggaagattcactagtgcagaaatggggagagaaaggtgggaaaaggagaaaccactgatttcaaggcgttttggtcctgagggtaatttttaaaaataccgaaaccgaatagtggtgccgaagggatgaatcgttgaaaacggagattctagaggaagcccatcgatccaagtatacaatccatccgggtagtagcaagatgtatcaggacctgaaaaggaacctattggtgggacaatatgaagaaggaaatcgctctgtacgtgcaaaaatgtctcgtttgccaacaggttaaagcggagcatcaaaaaccatcgggcttgttacagccccttgagatacccgagtggaagtggggaaaacatcaccatggacttcgtttcaggtttgccgcggacgcaaagaggacacgcatgccgtttgggtgatcgtcgatcgattaaccaaatcggcccatttcttgccagtaaacatgaagtattccatggacaagtttggcccaactgtacatggaggcgagatcgtaaggctacacggcgttccagtcagcatcgagtcagcatcggtctccgatcgagatccaccgtttcgtatctcgattttggcagaaattccaagaaactctagggacgaaactcaacttgagcacgacctatcaccctcagacggatggacaatcggagcgaacaatccttaaactctcgaggacatgctacggacttgcattctggattttggaggcaactggggtcaacacatgaccttagtagagtttgcatacaacaacagctaccattcgtcgattcaaatggctccatatgaagcactatagtggacgaaaatgccgatcaccaatttactgggatgaagttggcgagaggaaggcattagatccaaccactattccatgggatggaagaggctcgagaaaaggtcaagttgatacggcaacagactccaaactgctcaaagccgacaaaagagctacgcagacaaccgaagaaaagacttggagttcgaagttggagaccgtgttttcctcaaggttacaccgttacggagcgtcacggcgggtaaaggaaagaaattccaaccgaggttcgttggaccctacaagatcctccagagggtaggaacggtcgcgtatcgattagagctgccgtccagtctctctcgaattcacgacgtcttccacgtctcgatgctcaagaagtactatcccgacccatcccatatcttacaaccagaggagatcgacgtagacgaatcgcttgcttacgaagaagaaaacctgtccaagtgcttgaccggaaagtcaaagagcctcagaaacaagcagattccgttagtgaaagtactatggagaaaccaatggggtagaggaagctacctgggaagtggaagaagaaatgcgaaagaagtaccccaaatcttttcgggaattaaggtgagaaaatttcgaggacgaaatttcttttagtgaattggaagatgaagaatttcgacgacgaaatacgctaagggggagagagagtgtgagaacccgtaaaaatcccttaataattttcccaggggtttttatttcctttgagaacccgttaaaaaccctaataatttttcccgagggtttatattcccctttaatagcatgttttctgcatttcctggcttaggaattttcctgaggaattttatgagtaattatagttttagtatgatttttcttagccttggagagtttttggtaaaattaagagtatataatggacgtgggacccacaagtgcgaaaagttcggaaaaaaattcggccaattaggttaagtttcggatactgtgtaaaatttatcgggtgtttaagagataagtagagtgtgtgaagtgattgatgtgagaggaaaaaaaaagaaaaaaaagataagaatgcattaatgagtgtgacaagtgtcactctctgattggtttttactttaagattttactattccaattttttgacttttttgaacaaaggttaataaatatcttaaaaattgcccaaaaatcaccatttcttacctttctggatggccgggccctctctagcaagaagaaagacaaaaactcttcaacatcttggcaaattccctagctcaaatcatccaaactacttgcctaaatttagtttttactccataaaatttccttccttggggtgctagtaggtagttttagtgaagtttgtttgaagagctaaaggtgtccaacatctctatctctcttggttactaggtgagtggtgtgtgaatttcccctcttgcactttaatgaagcttaagttatgcttattagtgattaaagtggctgaaatttctggttttcacatcttggtttgaagtgatttgttggtgtaagtttttattttattgatgatttttctggtttaattggattatgatggttgtggttgtttaatgatgattgatacatatgaggggttatgactctagtggatggaaatgattgataatttgcaaccaattttgggatttgggaattaaattttgggaaagttagggttcatgggaagctacattctgcccgaatttattgcaccatgttagaggccgaattggccaacttggcttaaaaacatgaaaagttgtaggtattgatgtgtttgaagtgcctgcaaaaatttcaggtcatttggattagtgtagagtgagatatgtcgattttactgtttgctgttctgggttgatcagaatgcgaaaactgcacttgtaattggccattttttgactggaattgcttttggattttggatgttggtgtaacttctgatgaaatgtagcttgaatgtcttagctaccatatgccttttggaatcaatgcatttggacctgtatttagacttgagttgtactaattacagcattgtgttgatttgttaaacctgcaattaacggttctggttttggtattctgcatttttggacactagttgtgctacggatttggaactgagtggccttcgtacatttgttgtagccctgtcttttagcttacgagaatggttcgagatggtgggtccttacaccctcatccgcataagtcgtagcgcattttgtgcccattacccgcattaggaggccaaaaactgttttttgtgtttgggccaaaatgagttacattttcctgattttctggtttgctataatgctggtatatgtttgcaaaaccctatttgggggttgtgattggcattgctttatgactcgttatcgagtctcattgtactttgcttacgtgttcgtagggcgtgacggtggttcacgacattctcctgacggagaagtgcatgaaatagcacttaattgattggtgagtatactactcacttaattaatgttacaatgtggcttttggtatatgtgaatttgatgccttgaaggctaattatggctattgaaaatagattgcggttggagggtggtacttgaccgccctcaccccccttcgtgattccattcatgactgttttaccgttttacttgaaatactgcacttggctatatgagatattgaattccattcatgaaaaactagatttggtgtcatttgggacgaatgtccaacggccttactgtattactgagctcaatcccgtaggtaggttgtcaattgaatcgagccggcgagggttttggtcgtgaaaaattgacatgccacgggggactgtactggggaaccttgtagtaatgagactcttgattccggtaaacctcgagtattaccaaaagtgactgaatggagtgcgggcccggttggggtatggtttggtggaaggaatggaagtgaagtgaggtgctacgggttgggtattcttaaacaattgacggagggtcaatgaggttggatcaagaatgtaagcgtggaaatgggattgggctctttgagagccgtccgtatccttttaccgacttgtttaattctcactccttaattgtgtacttgaaatgaaaggttatgctaaaggcaccttgttgctcttatatgctcttcgatgcttatgtggtagtaactcattgagcttaacctcacttccgttccatttgttttccttacaggaaaaaatgactattttgggaaaggttgtggctagttggttgtcaagttgagctcatgtaaatgtatattttgaatagctccgtgcttgagggtgaaaaccctaagtgttttgtttccaaccaatggttggtttgtaatttggctaattgcacatgtatgaacttgttggatattttggtatgccgctttgggcttgtaaatgttaaatttcgatgtttatatatgttggattgatgtttggttgaaactccggattctgacgtggccggcactgttcatcatcggcttcgattttcaattttttttaaatttatgttgtgattttgacttgtttgcgtcgcgttggtgtgttccggaaacgtagtagatcgacgtaaactgatccgttagtcctggcgagagctgggcagggcagtccgctaacccctttggttcgccttaggggaaggtggggctgttacaaca

SEQ ID NO: 2 - DNA, 3042 bp, Coffea canephora
tgtgagaacccgtaaaaatcccttaataattttcccaggggtttttatttcctttgagaacccgttaaaaaccctaataatttttcccgagggtttatattcccctttaatagcatgttttctgcatttcctggcttaggaattttcctgaggaattttatgagtaattatagttttagtatgatttttcttagccttggagagtttttggtaaaattaagagtatataatggacgtgggacccacaagtgcgaaaagttcggaaaaaaattcggccaattaggttaagtttcggatactgtgtaaaatttatcgggtgtttaagagataagtagagtgtgtgaagtgattgatgtgagaggaaaaaaaaagaaaaaaaagataagaatgcattaatgagtgtgacaagtgtcactctctgattggtttttactttaagattttactattccaattttttgacttttttgaacaaaggttaataaatatcttaaaaattgcccaaaaatcaccatttcttacctttctggatggccgggccctctctagcaagaagaaagacaaaaactcttcaacatcttggcaaattccctagctcaaatcatccaaactacttgcctaaatttagtttttactccataaaatttccttccttggggtgctagtaggtagttttagtgaagtttgtttgaagagctaaaggtgtccaacatctctatctctcttggttactaggtgagtggtgtgtgaatttcccctcttgcactttaatgaagcttaagttatgcttattagtgattaaagtggctgaaatttctggttttcacatcttggtttgaagtgatttgttggtgtaagtttttattttattgatgatttttctggtttaattggattatgatggttgtggttgtttaatgatgattgatacatatgaggggttatgactctagtggatggaaatgattgataatttgcaaccaattttgggatttgggaattaaattttgggaaagttagggttcatgggaagctacattctgcccgaatttattgcaccatgttagaggccgaattggccaacttggcttaaaaacatgaaaagttgtaggtattgatgtgtttgaagtgcctgcaaaaatttcaggtcatttggattagtgtagagtgagatatgtcgattttactgtttgctgttctgggttgatcagaatgcgaaaactgcacttgtaattggccattttttgactggaattgcttttggattttggatgttggtgtaacttctgatgaaatgtagcttgaatgtcttagctaccatatgccttttggaatcaatgcatttggacctgtatttagacttgagttgtactaattacagcattgtgttgatttgttaaacctgcaattaacggttctggttttggtattctgcatttttggacactagttgtgctacggatttggaactgagtggccttcgtacatttgttgtagccctgtcttttagcttacgagaatggttcgagatggtgggtccttacaccctcatccgcataagtcgtagcgcattttgtgcccattacccgcattaggaggccaaaaactgttttttgtgtttgggccaaaatgagttacattttcctgattttctggtttgctataatgctggtatatgtttgcaaaaccctatttgggggttgtgattggcattgctttatgactcgttatcgagtctcattgtactttgcttacgtgttcgtagggcgtgacggtggttcacgacattctcctgacggagaagtgcatgaaatagcacttaattgattggtgagtatactactcacttaattaatgttacaatgtggcttttggtatatgtgaatttgatgccttgaaggctaattatggctattgaaaatagattgcggttggagggtggtacttgaccgccctcaccccccttcgtgattccattcatgactgttttaccgttttacttgaaatactgcacttggctatatgagatattgaattccattcatgaaaaactagatttggtgtcatttgggacgaatgtccaacggccttactgtattactgagctcaatcccgtaggtaggttgtcaattgaatcgagccggcgagggttttggtcgtgaaaaattgacatgccacgggggactgtactggggaaccttgtagtaatgagactcttgattccggtaaacctcgagtattaccaaaagtgactgaatggagtgcgggcccggttggggtatggtttggtggaaggaatggaagtgaagtgaggtgctacgggttgggtattcttaaacaattgacggagggtcaatgaggttggatcaagaatgtaagcgtggaaatgggattgggctctttgagagccgtccgtatccttttaccgacttgtttaattctcactccttaattgtgtacttgaaatgaaaggttatgctaaaggcaccttgttgctcttatatgctcttcgatgcttatgtggtagtaactcattgagcttaacctcacttccgttccatttgttttccttacaggaaaaaatgactattttgggaaaggttgtggctagttggttgtcaagttgagctcatgtaaatgtatattttgaatagctccgtgcttgagggtgaaaaccctaagtgttttgtttccaaccaatggttggtttgtaatttggctaattgcacatgtatgaacttgttggatattttggtatgccgctttgggcttgtaaatgttaaatttcgatgtttatatatgttggattgatgtttggttgaaactccggattctgacgtggccggcactgttcatcatcggcttcgattttcaattttttttaaatttatgttgtgattttgacttgtttgcgtcgcgttggtgtgttccggaaacgtagtagatcgacgtaaactgatccgttagtcctggcgagagctgggcagggcagtccgctaacccctttggttcgccttaggggaaggtggggctgttacaaca

SEQ ID NO: 3 - DNA, 20 bp, Artificial Sequence
  primer
gttagtcctggcgagagctg

SEQ ID NO: 4 - DNA, 20 bp, Artificial Sequence
  primer
aagccaggaaatgcagaaaa

(19) United States
(12) Patent Application Publication
(10) Pub. No.: US 2015/0044677 A1
(43) Pub. Date: Feb. 12, 2015

(54) CLEM2, ACTIVE RETROTRANSPOSON OF COFFEE PLANTS

(71) Applicant: INSTITUT DE RECHERCHE POUR LE DÉVELOPPEMENT (IRD), Marseille (FR)

(72) Inventors: Alexandre De Kochko, Montpellier (FR); Perla Hamon, Teyran (FR); Clemence Hatt, Montpellier (FR); Valerie Poncet, Montpellier (FR); Serge Hamon, Teyran (FR); Romain Guyot, Montpellier (FR); Christine Tranchant-Dubreuil, Saint Andre De Sangonis (FR)

(21) Appl. No.: 14/388,040

(22) PCT Filed: Apr. 5, 2013
(86) PCT No.: PCT/EP2013/057206

§ 371 Date: Sep. 25, 2014

(30) Foreign Application Priority Data
Apr. 6, 2012 (FR) 12/53239

(51) Int. Cl. C12Q 1/68; C12N 15/11

(52) U.S. Cl. CPC C12Q 1/6895; C12N 15/11; C12Q 2600/156; C12Q 2600/13
USPC 435/6.11; 536/23.1; 536/24.33

(57) ABSTRACT

The present invention relates to Clem2, the first active LTR retrotransposon identified in the coffee plant, and its use in the clonal and/or varietal identification of coffee plants. The invention provides PCR primers, kits comprising such PCR primers, and methods using such kits and/or PCR primers for the clonal and/or varietal identification of several coffee species. The primers, kits and methods are particularly useful for coffee certification and traceability.

### RELATED APPLICATIONS

The present application claims priority to French Patent Application number FR 12 53239 filed on Apr. 6, 2012. The content of the French Patent Application is incorporated herein by reference in its entirety.

### FIELD OF THE INVENTION

The present invention relates to the identification of varietal and/or clonal species of the Coffea genus, allowing, in particular, the traceability of commercial coffees.

### CONTEXT OF THE INVENTION

The importance of coffee in the world economy cannot be underestimated. It is one of the basic products that are the most widely traded throughout the world. Coffee is, immediately after oil, the major source of currency for developing countries. The growing, transformation, transport and marketing of coffee employ millions of people throughout the world. Coffee is grown in approximately 80 countries, occupying more than 10.2 million hectares of land in the tropical and subtropical regions of the planet, in particular in Africa, in Asia and in Latin America. For several years, the annual world production of coffee has exceeded six million metric tons, and this production is ever increasing.

Although the Coffea genus includes more than 100 species, the two coffee species which are the most important from an economic point of view are Coffea arabica (Arabica coffee)—which represents more than 60% of world production—and Coffea canephora (Robusta coffee). These two species have very different characteristics. C. arabica grows mainly in elevated regions and is very sensitive to most coffee diseases and parasites. The coffee produced from this species is generally more appreciated by connoisseurs due to its aroma and its low caffeine content. C. canephora, for its part, gives a more bitter, less aromatic drink with higher caffeine content. C. canephora is adapted to the low-altitude humid regions of Africa, Latin America and Asia. Its culture requires less attention than C. arabica. The differences in properties (growing properties and especially taste properties) which influence the differences in price do not only exist between coffee species, but also in terms of varieties and origins within one and the same species.

Today, the genetic diversity of plant species is studied using molecular markers which, in contrast to the markers associated with morphological, physiological or biochemical characteristics directly reveal genetic inheritance modifications, whether or not they are reflected by a phenotypic modification. Most molecular labelling strategies detect mutations in the DNA sequence, such as for example AFLP (Amplified Fragment-Length Polymorphism) markers or SNP (Single Nucleotide Polymorphism) markers, or modifications in the number of copies of very short repeat units of microsatellite type, such as SSR (Simple Sequence Repeat) markers. As shown by the studies carried out on the coffee plant (Anthony et al., Theor. Appl. Genet., 2002, 104: 894-900; Steiger et al., Theor. Appl. Genet., 2002, 105: 209-215; Vieira et al., Genet. Mol. Biol., 2010, 33: 507-514), these strategies make use of a set of several markers with a discriminatory capacity at the clonal or varietal level that is limited due to their low genomic coverage.

A third category of modifications of the genetic inheritance is linked to the presence and the activity of mobile DNA sequences known as “transposable elements”. These sequences are capable of moving in the genome and of inserting therein at various places, thus creating an insertion polymorphism. Among the various transposable elements, LTR (Long Terminal Repeat) retrotransposons appear to be powerful tools for understanding genome dynamics and evaluating genetic diversity in plants (Kumar et al., Annu. Rev. Genet., 1999, 33: 479-532; Kumar and Hirochika, TRENDS in Plant Science, 2001, 6: 127-134). However, only three coffee LTR retrotransposons are currently known, all three having been identified by the present inventors. The retrotransposons Nana and Divo (Hamon et al., Mol. Genet. Genomics, June 2011, 285: 447-460) were identified in C. canephora, and the retrotransposon CART No. 109 (Yu et al., Plant J., July 2011, 67: 305-317) was identified in C. arabica. However, these three retrotransposons are defective and inactive.

Despite the progress made, which has enabled a better understanding of the genetic diversity of coffee plants, there is still a need in the art for simple, rapid and effective methods for individual clonal and/or varietal identification in the various coffee species, in particular in C. canephora and C. arabica. Having such methods is all the more important since coffee production and the coffee trade are increasingly subject to certification and/or labelling programmes.

### SUMMARY OF THE INVENTION

The present invention generally relates to active LTR retrotransposons (LTR-RTNs) identified in coffee plants and concerns the use of these retrotransposons as molecular markers of genetic diversity in coffee plants. More specifically, the present inventors have found, surprisingly, that certain LTR-RTNs, including in particular the element Clem2, in contrast to the vast majority of retrotransposons identified in plants, are transcriptionally active in C. canephora in the absence of an external factor (biotic or abiotic stress), irrespective of the developmental stage of the coffee plant and independently of the plant tissue or organ considered. Furthermore, the abundance of Clem2 was found to be exceptionally high in the C. canephora genome.

The unusual properties of Clem2 (exceptional abundance and transcriptional activity) make it a particularly effective molecular marker for determining intraspecific polymorphism in the coffee plant, as demonstrated by the present inventors.

Consequently, in a first aspect, the present invention relates to an isolated active LTR retrotransposon of the Clem2 family having a nucleotide sequence comprising, at each of its 5′ and 3′ ends, the sequence set forth in SEQ ID NO: 2 or a sequence consisting of 3042 nucleotides and having at least 80% homology, preferably at least 85% homology, and even more preferably at least 90% homology, with SEQ ID NO: 2.

In certain embodiments, the isolated active LTR retrotransposon of the Clem2 family hybridizes to SEQ ID NO: 1, or to the sequence complementary to SEQ ID NO: 1, under stringent hybridization conditions or moderately stringent hybridization conditions.

In certain embodiments, the active LTR retrotransposon of the Clem2 family is isolated from a coffee plant and/or is identical to an active LTR retrotransposon present in the genome of a coffee plant.

The invention also relates to a primer comprising an oligonucleotide containing between 15 and 35 consecutive nucleotides:
of the nucleotide sequence set forth in SEQ ID NO: 2, or
of the sequence complementary to SEQ ID NO: 2, or
of a nucleotide sequence consisting of 3042 nucleotides and having at least 80% homology, preferably at least 85% homology, even more preferably at least 90% homology, with the sequence set forth in SEQ ID NO: 2, or
of the sequence complementary to a nucleotide sequence consisting of 3042 nucleotides and having at least 80% homology, preferably at least 85% homology, even more preferably at least 90% homology, with the sequence set forth in SEQ ID NO: 2.

The oligonucleotide can, for example, have the sequence set forth in SEQ ID NO: 3 or the sequence set forth in SEQ ID NO: 4.

In certain embodiments, a primer according to the invention also comprises a detectable label and/or a sequence of 1, 2, 3 or 4 random bases.

The invention also relates to the use of primers as described herein for the varietal and/or clonal identification of a coffee plant.

The present invention also relates to a method for varietal and/or clonal identification of a coffee plant, comprising a step of: establishing the insertion profile of an active LTR retrotransposon of the Clem2 family in the genome of the coffee plant tested. Establishing the insertion profile of an active LTR retrotransposon of the Clem2 family comprises determining insertion sites of the retrotransposon in the genome of the coffee plant.

In a method for varietal and/or clonal identification according to the invention, the insertion profile can be established using the SSAP (Sequence-Specific Amplification Polymorphism) technique, the REMAP (Retrotransposon-Microsatellite Amplified Polymorphism) technique, the IRAP (Inter-Retrotransposon Amplified Polymorphism) technique, the RBIP (Retrotransposon-Based Insertion Polymorphism) technique, or any variation thereof.

In certain embodiments, a method according to the invention is characterized in that it is carried out on total DNA extracted from a sample of the coffee plant tested, where the sample of the coffee plant is a sample of protoplast, organ, callus, seed, flower, fruit, leaf, stem, root, cutting or bean of the coffee plant tested. When the identification test relates to a coffee bean, the method can be carried out on a green coffee bean or a roasted coffee bean.

In certain embodiments, the step of establishing the insertion profile of the active LTR retrotransposon comprises amplifying total DNA extracted from a sample of the coffee plant tested using at least one primer of the invention, thus generating fragments that are specific of the insertion of the retrotransposon in the genome of the coffee plant tested.

In certain embodiments, a method according to the invention further comprises comparing the insertion profile obtained for the coffee plant tested with the insertion profile of the same active LTR retrotransposon in the genome of a control coffee plant of the same variety or of the same clone as the coffee plant tested. In certain preferred embodiments, the coffee plant tested and the control coffee plant both belong to the C. canephora species or to the C. arabica species.

The invention also provides a kit for the varietal and/or clonal identification of coffee plants, comprising at least one primer according to the invention and instructions for carrying out a method according to the invention.

These and other objects, advantages and features of the present invention will become apparent to those of ordinary skill in the art having read the following detailed description of the preferred embodiments.

### BRIEF DESCRIPTION OF THE DRAWING

FIG. 1 is an electrophoresis gel (1% agarose/1×TBE), migrated at 100V, showing the PCR or RT-PCR amplification profiles obtained with the gDNA, the cDNA and the RNA of various genotypes of C. canephora and C. arabica.

FIG. 2 is an electrophoresis gel (1% agarose/1×TBE), migrated at 100V, showing the PCR amplification profiles obtained with the gDNA and RNA of BA53 and BD55 using the pairs of primers remap and RT-clem2, and g3 (gene present on the BAC 46C02 and acting as a positive control).

FIG. 3(A) is an electrophoresis gel (1% agarose/1×TBE), migrated at 100V, showing the PCR amplification profiles obtained with the BA53 and BD55 genotypes of C. canephora using the pair of primers remap-clem2 designed in the LTR regions so as to demonstrate the circular form of the Clem2 RTN. The g3 primers were used as a control. FIG. 3(B) is a 1% agarose/1×TBE electrophoresis gel, migrated at 100V, showing the PCR amplification profiles obtained with the BA53 genotype of C. canephora using the pair of primers RT-clem2 designed in the reverse transcriptase (RT) domain of the Clem2 RTN.

FIG. 4 is an electrophoresis gel (2% agarose) showing the insertion profile of Clem2, established using the TRAP technique, for two genotypes (BA53 and BD55) of C. canephora. Some polymorphic bands are encircled in white.

FIG. 5 shows the results of in situ hybridization of chromosomes at the metaphase stage of C. canephora using the Clem2 retrotransposon (top photo) and the Clem5 retrotransposon (bottom photo). The green labelling represents the retrotransposons, the blue labelling the chromosomes and the red labelling the 18s control.

### DETAILED DESCRIPTION OF THE INVENTION

As mentioned above, the present invention relates to active LTR retrotransposons of the coffee plant and to the use thereof in clonal and/or varietal identification in various species of coffee plant, in particular the C. canephora and C. arabica species. The invention relates in particular to the active LTR retrotransposon, Clem2.

### I—Clem2 and Derived Sequences

As indicated above, Clem2 is an active LTR retrotransposon. As used herein, the term “LTR retrotransposon” refers to a retrotransposon (i.e., a class I transposable element, which copies itself and inserts itself via an RNA intermediate by virtue of an enzymatic machinery encoded by the element itself) comprising, at each of its 5′ and 3′ ends, a long terminal repeat (or LTR) region. LTR retrotransposons move in the genome according to the “copy-and-paste” model. They are transcribed into RNA by the cell machinery, and then, after migration in the cytoplasm, they reverse-transcribe a complementary DNA (cDNA) from their mRNA in virus-like particles of which they encode the capsid subunits. A second DNA strand is synthesized after elimination of the template RNA, and then, after returning to the nucleus, they incorporate this DNA at a new chromosomal locus.

Most retrotransposons identified in plants are transcriptionally inactive (Kumar et al., Annu. Rev. Genet., 1999, 33: 479-532) or active only during particular steps of the plant development or in response to a biotic or abiotic stress (Hirochika, EMBO J., 1993, 12: 2521-2528; Kumar and Hirochika, TRENDS in Plant Science, 2001, 6: 127-134). LTR retrotransposons have thus been identified in many plants, such as Tto1 (Hirochika, EMBO J., 1993, 12: 2521-2528), Tnt1 (Grandbastien, Trends Plant Sci., 1998, 3: 181-189) and Tnp2 (Hirochika et al., Gene, 1995, 165: 229-232) in tobacco, Tos17 in rice (Hirochika et al., PNAS USA, 1996, 93: 7783-7788), BARE-1 in barley (Manninen et al., Plant Mol. Biol., 1993, 22: 829-846) or CIRE-1 in the orange tree (Rico-Cabanas et al., Mol. Genet. Genomics, 2007, 277: 365-377), but the transcriptional activity of these retrotransposons has been observed only under conditions of stress or only in a limited number of tissues (Grandbastien, Trends Plant Sci., 1998, 3: 181-189). The present inventors have found, surprisingly and unexpectedly, that certain LTR-RTNs, including Clem2, are transcriptionally active in C. canephora in the absence of stress, irrespective of the developmental stage of the coffee plant and independently of the plant tissue or organ considered. Thus, the term “active LTR retrotransposon”, as used herein, refers to an LTR retrotransposon with a transcriptional activity that is (1) independent of the presence of an external factor, such as a biotic or abiotic stress, (2) omnipresent in the tissues and organs of the coffee plant, and (3) continuous during the development of the coffee plant.

Furthermore, a preliminary study carried out by the inventors has shown that the abundance of Clem2 in the C. Canephora genome is exceptionally high. In comparison, the abundance of other LTR retrotransposons identified so far in the coffee plant (Nana and Divo) is between 3 and 6 times lower.

The unusual properties of certain LTR-RTNs such as Clem2 (exceptional abundance and transcriptional activity) make them particularly effective molecular markers for not only determining interspecific polymorphism in coffee plants but also, and more importantly, for determining intraspecific polymorphism in coffee plants. Indeed, the high abundance of these elements in the genome of coffee plants facilitates the detection of such insertion polymorphisms. Furthermore, owing to their transcriptional activity, they are capable of generating differences between contemporary genotypes by inserting themselves at new sites of the genome.

The present inventors have established the sequence of Clem2. The nucleotide sequence of Clem2 comprises 11839 nucleotides. Consequently, in a first aspect, the present invention relates to the isolated active LTR retrotransposon Clem2, which consists of the nucleotide sequence set forth in SEQ ID NO: 1.

The terms “nucleotide sequence”, “nucleic acid”, “nucleic sequence”, “polynucleotide” and “oligonucleotide” are used here interchangeably. They refer to a given sequence of nucleotides, modified or not, which defines a region of a nucleic acid molecule and which may be either under the form a single strain or double strain DNAs or under the form of transcription products thereof. The term “isolated”, as used herein in reference to a polynucleotide, refers to a polynucleotide which, by virtue of its origin or its manipulation, is separated from at least some components with which it is naturally associated. Alternatively or additionally, the term “isolated” is intended to mean a polynucleotide which is produced or synthesized by man.

The present invention also relates to an isolated nucleic acid consisting of the sequence complementary to SEQ ID NO: 1. As used herein, the expression “sequence complementary to” a given nucleotide sequence refers to a sequence which forms, by hybridization, a stable duplex with said nucleotide sequence. The term “complementary sequence” denotes both the complementary sequence presented in the 3′→5′ direction and the complementary sequence presented in the 5′→3′ direction (i.e., the reverse complementary sequence). The term “hybridization”, as used herein, refers to the head-to-tail association of two single-stranded polynucleotides by Watson-Crick pairings (A-T, G-C). In certain cases, the hybridization is perfect, i.e., the sequences are totally complementary. Thus, for example, the term “the sequence complementary to SEQ ID NO: 1” is the nucleotide sequence which is perfectly or totally complementary to SEQ ID NO: 1. In other cases, the hybridization is imperfect, i.e., the sequences are not totally complementary but are sufficiently complementary to hybridize to one another and to form a double-stranded structure under stringent hybridization conditions. Thus, for example, “a sequence complementary to SEQ ID NO: 1” is a nucleotide sequence which forms, by hybridization, a stable duplex with SEQ ID NO: 1, but which is not necessarily perfectly complementary to SEQ ID NO: 1.

Clem2, as defined by the sequence SEQ ID NO: 1, comprises, at each of its 5′ and 3′ ends, a long terminal repeat (LTR) region, having the nucleotide sequence set forth in SEQ ID NO: 2 (which consists of 3042 nucleotides). The present invention therefore also relates to an isolated nucleic acid molecule consisting of the nucleotide sequence SEQ ID NO: 2 or the sequence complementary to SEQ ID NO: 2.

Clem2, as defined by the sequence SEQ ID NO: 1, is the representative of a family of active LTR retrotransposons. This family comprises all the retrotransposons which have identical or very similar (in sequence and in length) LTRs. Furthermore, several transposable elements belonging to the same family do not have exactly the same sequence owing to the fact that, after their insertion, these elements evolve and accumulate mutations. However, it is generally admitted in the art that transposable elements which have LTR regions of the same length and exhibiting a sequence identify of at least 80% belong to the same family.

Consequently, the present invention also relates to any isolated active LTR retrotransposon of the Clem2 family, which has a sequence comprising two identical long terminal repeat (LTR) regions: each LTR region consisting of 3042 nucleotides and having at least 80% sequence homology, preferably at least 85% sequence homology, even more preferably at least 90% sequence homology, with the sequence set forth in SEQ ID NO: 2. Preferably, such an active LTR retrotransposon of the Clem2 family is isolated from a coffee plant (i.e., is identical to an active LTR retrotransposon present in the genome of a coffee plant). Using the sequences of the LTR regions of Clem2, those skilled in the art know how to isolate any retrotransposon of the Clem2 family present in the genome of a coffee plant. The present invention also relates to any nucleic acid molecule consisting of the nucleotide sequence complementary to the sequence of an active LTR retrotransposon of the Clem2 family.

In certain embodiments, an isolated active LTR retrotransposon of the Clem2 family according to the invention has a nucleotide sequence that is homologous to the sequence SEQ ID NO: 1. As used herein, the expression “nucleotide sequence homologous to the sequence SEQ ID NO: 1” refers to any nucleotide sequence which differs from the sequence set forth in SEQ ID NO: 1 by substitution, deletion and/or insertion of one nucleotide or of a limited number of nucleotides, at positions such that these homologous nucleotide sequences are active LTR retrotransposons of the Clem2 family. Preferably, a nucleotide sequence homologous to the sequence set forth in SEQ ID NO: 1 has a percentage identity of at least 90%, preferably of at least 95% (for example 96%, 97%, 98% or 99%), with the sequence SEQ ID NO: 1.

The term “percentage identity” or “homology” between two nucleotide sequences is intended to denote a percentage of nucleotides which are identical between the two sequences to be compared, obtained after optimal alignment. This percentage is purely statistical and the differences between the two sequences are distributed randomly and over the entire length of the sequence. The terms “optimal alignment” and “best alignment”, which are used interchangeably here, denote the alignment for which the percentage identity determined as described below is the highest. The optimal alignment of the sequences, required for the comparison, can be produced manually or by means of computer programs (GAP, BESTFIT, BLASTP, BLASTN, FASTA and TFASTA, which are available, for example, either on the NCBI website or in the Wisconsin Genetics Software Package, Genetics Computer Group, Madison, Wis.). The percentage identity between two nucleotide sequences is calculated by determining the number of identical positions for which the nucleotide is identical between the two sequences, by dividing this number of identical positions by the total number of positions compared, and by multiplying the result obtained by 100.

Preferably, in the context of the present invention, a nucleotide sequence homologous to SEQ ID NO: 1 hybridizes specifically to the sequence complementary to the sequence set forth in SEQ ID NO: 1 under stringent hybridization conditions or moderately stringent hybridization conditions. Said hybridization conditions can be established by means of conventional protocols described, for example, in Sambrook et al., “Molecular Cloning—A Laboratory Manual”, Cold Spring Harbor Laboratory Press, 1989, or Ausubel et al., “Current Protocols in Molecular Biology”, Green Publishing Associates and Wiley Interscience, 1989.

II—Use of Clem2 in the Varietal and/or Clonal Identification of Coffee Plants

As demonstrated by the present inventors, Clem2 allows varietal and/or clonal identification in various species of coffee plants. This identification is carried out by comparing the insertion profiles of Clem2 in the genome of various coffee plants.

### Coffee Plant Samples

In a method according to the invention, the step of establishing the insertion profile of Clem2 in the genome of a coffee plant is carried out on a sample of the coffee plant to be tested, and preferably on total DNA extracted from the coffee plant sample.

Any coffee plant sample containing genomic DNA can therefore be used in the practice of the present invention. For example, genomic DNA can be extracted from coffee plant protoplasts, organs, calluses, seeds, flowers, fruits (called “cherries”), leaves, stems, roots or cuttings. Genomic DNA can also be extracted from coffee beans, whether they are green beans (i.e., beans obtained by extraction of the cherries) or roasted beans (i.e., beans obtained by torrefaction, the operation which consists in roasting the coffee beans).

Methods for extracting DNA from biological tissues are well known in the art (see, for example, Sambrook et al., “Molecular Cloning—A Laboratory Manual”, Cold Spring Harbor Laboratory Press, 1989). Several kits are also commercially available (for example from BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.)), which can be used to extract the DNA from coffee plant samples.

### Techniques for Determining the Insertion Profiles of Clem2

As those skilled in the art will recognize, in the practice of the invention, the insertion profile of an LTR-RTN, such as Clem2, in the genome of a coffee plant can be determined by any appropriate technique known in the art, since the technique used is not a limiting factor of the invention.

The insertion profile of a transposable element is commonly generated by PCR (Polymerase Chain Reaction) amplification of a collection of fragments of insertion borders (border region containing the end of the transposable element and the beginning of the genomic sequences of the host that are flanking the insertion). These fragments start in the terminal regions (LTR regions for an LTR retrotransposon) of the various copies of the transposable element and end either in the genomic DNA flanking the transposable element or in the terminal region of other copies of the transposable element. The collection of amplified fragments reflects the various insertion sites that exist in the genotype tested and forms a “map” or “pattern” of the representation of the transposable element in this genotype. The term “insertion profile of Clem2”, as used herein, refers to the map or pattern of the representation of Clem2 (or of another isolated active retrotransposon of the Clem 2 family) in a given genotype.

In certain embodiments, determination of the insertion profile of Clem2 (or of another isolated active retrotransposon) is carried out using one of the methods known in the art under the names SSAP (Sequence-Specific Amplification Polymorphism), REMAP (Retrotransposon-Microsatellite Amplified Polymorphism), IRAP (Inter-Retrotransposon Amplified Polymorphism), or RBIP (Retrotransposon-Based Insertion Polymorphism). These methods are known to those skilled in the art and their use in the study of plant diversity has been described (see, for example, Kumar et al., Annu. Rev. Genet., 1999, 33: 479-532; Kumar and Hirochika, TRENDS in Plant Science, 2001, 6: 127-134; Mihri and Grandbastien, in “La Génomique en Biologie Végétale” [Genomics in Plant Biology], Eds. Morot-Gandry and Bria, INRA publications, Paris, 2004, pp. 377-401).

Briefly, in the SSAP technique (Waugh et al., Mol. Gen. Genetics, 1997, 253: 687-694), the amplified sequence borders or lines the retrotransposon. The first step consists in digesting the purified genomic DNA with two restriction enzymes, one which cleaves rarely (for example, EcoR1 or Pst1) and the other which cleaves more frequently (for example, Mse1 or Tru1). Linkers, of known sequence containing from 10 to 15 bases, are then added, by ligation, to the ends of the sites of cleavage of the genomic DNA by the restriction enzymes. A PCR amplification is then carried out with a primer corresponding to the sequence of the linkers and a primer corresponding to a sequence located at the end of an LTR of the retrotransposon oriented in such a way that the amplification is carried out towards the exterior of the LTR-RTN. The latter primer is labelled (radioactivity, fluorescence, or the like) so as to reveal only the fragments anchored in the retrotransposon. Each retrotransposon copy will thus generate an amplification fragment containing the terminal sequences of the retrotransposon and the flanking genomic sequences located between the retrotransposon and the restriction site. The amplified fragments are then separated according to their size by a high-resolution technique (for example, sequencing gel or capillary sequencer) so as to obtain an insertion profile of the retrotransposon. Since this approach calls for several steps (digestion, ligation, pre-amplification), it is more sensitive to a lack of repetitiveness.

The IRAP technique is based on the detection of insertion polymorphisms by direct PCR between two copies of the transposable element (in this case two copies of Clem2) which are sufficiently close to one another in the genome to allow amplification of the intermediate region (Kalendar et al., Theoret. Applied Genetics, 1999, 98: 704-711; Kalendar & Schulman, Nat. Protoc., 2006, 1: 2478-2484). The IRAP method is simpler to carry out than the SSAP technique, since it is not necessary to digest the DNA. The primers used in the IRAP technique correspond to sequences located at the end of the retrotransposon LTRs, with optionally a few added random bases, depending on the frequency of the element in the genome, in order to amplify sequences located between two close copies of the retrotransposon. For this, the primers are directed towards the exterior of the retrotransposon. The amplified products are generally resolved by high-resolution electrophoresis on agarose gel or preferentially on acrylamide gel.

The REMAP technique is conceptually similar to the TRAP technique. It is based on a direct PCR amplification between the transposable element (in this case the Clem2 retrotransposon) and microsatellites (Kalendar et al., Theoret. Applied Genetics, 1999, 98: 704-711; Kalendar & Schulman, Nat. Protoc., 2006, 1: 2478-2484). Micro satellites, which are particular DNA sequences characterized by the repetition of a nucleotide motif (generally of one to six nucleotides), are omnipresent in the genome of living organisms. The REMAP technique uses primers corresponding to sequences located at the end of the LTRs and directed towards the exterior of the retrotransposon, and primers of SSR (Simple Sequence Repeat) type containing a set of repeats and at least one random base in the 3′ position to be used for anchoring. Like TRAP, the REMAP technique is carried out on an undigested DNA, and the amplified products are resolved by high-resolution electrophoresis since the primers used are labelled.

In the RBIP technique (Flavell et al., Plant J., 1998, 16: 643-650), the presence of a transposable element (in this case the Clem2 retrotransposon) in a given site is tested by comparing the amplification of this site using a pair of primers surrounding it, with the amplification obtained using one of these primers combined with a primer specific for the sequence of the transposable element. An amplification obtained in both cases indicates the presence of the retrotransposon at the site tested, whereas an amplification obtained only with the first combination of primers indicates the absence of the retrotransposon at the site tested. If the primers are specific, simple agarose gel electrophoresis with staining of the amplification products with ethidium bromide or with another intercalating agent (for example, SYBR Green) is sufficient. This “site specific” method requires prior knowledge of the genomic sequences flanking the insertion. This information can be acquired, for example, by performing an SSAP with a primer anchored in the known flanking region, and carried out on a genotype where the site is empty. This information can also be obtained from databanks, by searching for complete insertions or insertion border regions. The main limitation of this technique is that it gives results only site-by-site, and not a pattern of the insertion polymorphism.

The present invention therefore describes a method for varietal and/or clonal identification of coffee plants, comprising a step of establishing the insertion profile of Clem2 (or of another active LTR retrotransposon of the Clem2 family) in the genome of the coffee plant tested. Preferably, the step of establishing the insertion profile is carried out using an SSAP, REMAP, IRAP or RBIP technique, and therefore comprises the use of PCR primers.

### Primers and Probes for Determining the Insertion Profile of Active LTR-RTNs

Starting from the nucleotide sequence of Clem2 (or of another active LTR retrotransposon), those skilled in the art are able to design primers suitable for the technique selected for establishing the insertion profile of Clem2 (or of another active LTR retrotransposon).

The terms “primer” and “PCR primer” are used here interchangeably and denote an oligonucleotide which is capable of acting as a starting point for the synthesis of an amplification product, when it is placed under suitable amplification conditions (for example, salt concentration, temperature and pH) in the presence of nucleotides and of a nucleic acid polymerization agent (for example a DNA polymerase). A primer according to the invention comprises an oligonucleotide advantageously containing between 5 and 50 nucleotides, preferably between 15 and 35 nucleotides, even more preferably between 20 and 25 nucleotides (for example 20, 21, 22, 23, 24 or 25 nucleotides). In certain embodiments, a primer can also comprise a short additional sequence (containing, for example, 1, 2, 3 or 4 random bases).

In certain embodiments, a primer according to the invention is labelled so as to allow its detection (and, consequently, detection of the amplification products or amplicons obtained by PCR). Various types of labelling known to those skilled in the art can be used (radioactive labelling, fluorescence, chemiluminescence, and the like). The term “labelled primer” is therefore intended to denote a primer which contains, or which is associated with or bonded (for example covalently) to, a detectable label, such as, in particular, a radioactive isotope, a molecule with fluorescent or luminescent properties, etc.

In some embodiments, a primer according to the invention is designed using the sequences of the LTR regions of retrotransposon Clem2, i.e. the LTR region located at the 5′ end and at the 3′ end of Clem2 and consisting of the nucleotide sequence set forth in SEQ ID NO: 2.

In other embodiments, a primer according to the invention is designed using the LTR sequences of an active LTR retrotransposon of the Clem2 family (i.e. identical LTR regions located at the 5′ and 3′ ends of the retrotransposon, each LTR region consisting of 3042 nucleotides and having at least 80% sequence homology, preferably at least 85% sequence homology, even more preferably at least 90% sequence homology, with the sequence set forth in SEQ ID NO: 2).

As indicated above, the IRAP, SSAP and REMAP techniques amplify genomic DNA regions located between the LTRs of two close copies of the retrotransposon, between an LTR of the retrotransposon and a restriction site, and between an LTR of the retrotransposon and a microsatellite, respectively. Consequently, in these techniques, the primers used are directed towards the exterior of the retrotransposon. Preferably, the primers are designed using the sequences which are at the end of the LTR regions, i.e.: in the 5′→3′ direction, using the initial/beginning (or 5′) portion of the first LTR and using the final/end (or 3′) portion of the second LTR.

Thus, in certain embodiments, a primer according to the invention comprises an oligonucleotide containing between 15 and 35 consecutive nucleotide, preferably between 20 and 25 consecutive nucleotides (for example 20, 21, 22, 23, 24 or 25 consecutive nucleotides):
of the sequence set forth in SEQ ID NO: 2, or
of a nucleotide sequence consisting of 3042 nucleotides and having at least 80% homology, preferably at least 85% homology, even more preferably at least 90% homology, with the sequence set forth in SEQ ID NO: 2,
of the sequence complementary to SEQ ID NO: 2, or
of the sequence complementary to a nucleotide sequence consisting of 3042 nucleotides and having at least 80% homology, preferably at least 85% homology, even more preferably at least 90% homology, with the sequence set forth in SEQ ID NO: 2.

In one particular embodiment, a primer according to the invention comprises an oligonucleotide of sequence SEQ ID NO: 3.

In another particular embodiment, a primer according to the invention comprises an oligonucleotide of sequence SEQ ID NO: 4.

In certain embodiments, a primer according to the invention further comprises 1, 2, 3, or 4 random bases.

In some embodiments, a primer according to the invention further comprises a label for the detection of the primer and of the amplicons generated. As indicated above, a detectable label can be a radioactive isotope, a molecule with fluorescent or luminescent properties, and the like. The label can be integrated into the oligonucleotide making up the primer, or associated with this oligonucleotide (for example by covalent bonding).

Primers according to the present invention can be prepared by any suitable method known in the art, in particular from conventional methods of oligonucleotide synthesis, such as solid-phase synthesis methods. The primers according to the invention can, for example, be prepared using an oligonucleotide synthesizer (such as those sold, for example, by Applied Biosystems or GE Healthcare). Likewise, methods for labelling oligonucleotides are known in the art.

The present invention relates to the primers described herein (or any other primer which can hybridize to SEQ ID NO: 2 or to the sequence complementary thereto) and also to the use thereof for establishing the insertion profile of Clem2 (or of another active LTR retrotransposon of the Clem2 family) in the genome of a coffee plant thereby allowing clonal and/or varietal identification of the coffee plant.

### Analysis of the Insertion Profile of an Active LTR-RTN and Interspecific/Intraspecific Polymorphism

In a method according to the invention, after the insertion profile of Clem2 (or of another active LTR retrotransposon) has been obtained for the coffee plant tested, this profile is compared to the insertion profile of Clem2 (or of another active LTR retrotransposon) obtained for a control coffee plant. Given that the methods of the invention make it possible to identify intraspecific polymorphism in the coffee plant, the coffee plant tested and the control coffee plant are necessarily of the same species. The term “control coffee plant”, as used herein, refers to a coffee plant of which the species is known, and of which the variety or the identity of the cone is known, and is believed to be that of the coffee plant or coffee tested.

Comparing the retrotransposon insertion profiles allows to determine whether the coffee plant sample tested belongs to the same variety or to the same clone as the control coffee plant (identical insertion profiles) or whether it belongs to a different variety or clone (different insertion profiles).

As will be recognized by one skilled in the art, comparing the insertion profile obtained for the coffee plant tested and the insertion profile obtained from the control coffee plant can be carried out by comparing the two complete insertion profiles or by verifying the presence or absence, in the insertion profile obtained for the coffee plant tested, of one or more particular characteristics of the insertion profile obtained for the control coffee plant (for example, presence, on a gel, of one or more bands which are distinctive of the clonal and/or varietal identity of the control coffee plant).

Varietal and/or Clonal Identification of Coffee Plants

As demonstrated by the present inventors, Clem2 allows varietal and/or clonal identification in various coffee plant species, i.e., in other words, it allows varieties and/or clones within one and the same coffee plant species to be distinguished and identified. The term “variety”, as used herein, refers to a wild-type variety or a cultivated variety: selected or hybrid. As used herein, the term “clone” refers to a variety resulting from a clone (by somatic embryogenesis, cuttings, grafting or any other means of vegetative reproduction).

In the practice of the present invention, the coffee plant species can be chosen from, without limitation, the cultivated species: C. arabica, C. canephora, C. liberica, and C. dewevrei, and the wild-type species: for instance C. excelsa, C. eugenioides, C. stenophylla, C. mauritiana, C. racemosa, C. congencis, C. neo-arnoldiana, C. abeokutoe, C. perrieri and C. dybowskii among the 125 species of the genus described to date.

In certain preferred embodiments, the insertion profile of Clem2 (or of another active LTR retrotransposon) is used for determining the variety of a coffee plant of the C. arabica species. In other preferred embodiments, the insertion profile of Clem2 (or of another active LTR retrotransposon) is used for determining the clone of a coffee plant of the C. canephora species.

C. Arabica and Varieties Thereof.

C. arabica is genetically different from the other coffee plant species: it is tetraploid (44 chromosomes) and self-fertile. Today there are more than 200 varieties of C. arabica.

The C. arabica varieties which can be identified using the insertion profile of Clem2 (or of another active LTR retrotransposon) in their genome include, but are not limited to, the varieties: Arusha (grown in the Meru region of Tanzania, and in New Guinea), Bergendal (grown in Indonesia), Sidikalang (grown in Indonesia), Blue Mountain (a natural mutation of Typica grown in Jamaica, in Kenya, in Hawaii and in New Guinea), Bourbon (grown on Réunion, and in Latin America), Caturra (which is a mutation of the Bourbon variety, grown in Central America and South America), Catuai (which is a hybrid of Mundo Novo and Caturra, grown in Latin America), Charrieriana (a new variety found in Cameroon), Colombian (grown in Colombia), Ethiopian Harar (grown in Ethiopia), Ethiopian Sidamo (grown in Ethiopia), Ethiopian Yirgacheffe (grown in Ethiopia), French Mission (grown in Africa), Guadeloupe Bonifieur (grown in Guadeloupe), Hawaiian Kona (grown in Hawaii), Jamaican Blue Mountain (grown in Jamaica and in Africa), K7 (a Kenyan selection of French Mission and of Bourbon), Mayaguez (a cultivar of Bourbon grown in Rwanda), Mocha (grown in Yemen), Mundo Novo (a hybrid between Bourbon and Typica, grown in Latin America), Orange Bourbon, Yellow Bourbon, Pacamara (a hybrid of Typica and Maragogipe grown in Latin America), Pacas (a natural mutation of the Bourbon variety, grown in Latin America), Pache Comum (a mutation of Typica discovered in Guatemala), Pache Colis (a hybrid between Pache Comum and Caturra, which is grown in Latin America), Panama (a highly prized variety, grown in Panama and in Costa Rica), Maragogipe (a mutation of Typica, grown in Latin America), Ruiri (grown in Kenya), 5795 (grown in India and in Indonesia), SL28 (grown in Kenya), SL34 (selected from the French Mission variety grown in Kenya), Sumatra Mandheling and Sumatra Lintong (grown in Somalia), Sulawesi Toraja Kalossi (grown in Indonesia), and Typica.

C. Canephora and Clones Thereof.

C. canephora is a self-sterile diploid. C. canephora comprises more than 50 different varieties. The most commercially important variety is Robusta, which is the most widespread variety, in particular in Africa (Ivory Coast, Cameroon, Uganda, etc.) and in Asia (Indonesia, India, etc.), Kouillou, Conilon, Gimé, and Niaouli.

In certain preferred embodiments, the insertion profile of Clem2 (or of another active LTR retrotransposon) is used for determining the identity of the cultivated clone of a coffee plant of the C. canephora species.

Examples of C. canephora clones include, but are not limited to, the clones B5/461, B11/107, J21/126, C6/182, M5/197, H 865, 200/Y1, HB, K 26, 503/149, 259/S/56, 1S/6, 477/J32/69, 505/B60/177, LD 1, NC 8, NC 1, B42, BA53, BD55 and HD200.

Coffee Certification and/or Labelling Programmes

A method according to the present invention can be used in any context (for example in the development of new coffee plant varieties, in maintaining and managing coffee plant collections, in growing coffee plants and/or in coffee production) and also at any time in the coffee production chain ranging from the activities of nurserymen (validation of the material sold for the plantation) to coffee merchants (precise identification of the variety purchased), via all the steps of the chain involving green coffee purchasers, coffee rosters, etc. . . .

Several coffee certification and verification programmes (such as Fairtrade Certification, Biological Certification, Rainforest Alliance Certification, “Bird Friendly” Certification by the Smithsonian Migratory Bird Centre, and UTZ Certification) have been set up, mainly over the last ten years. These programmes define the criteria for socially, ecologically and economically responsible coffee production, also called “sustainable coffee production”. While the main objective of these certification programmes is to fight poverty in coffee-producing countries, they also offer the consumer the guarantee of a quality coffee of known origin resulting from a sustainable economy. Thus, the particularity of certified coffees is that they are “traceable”.

A method according to the invention for clonal and/or varietal identification of various species of the Coffea genus can therefore be used in coffee certification and verification programmes.

III—Kits for Clonal and/or Varietal Identification of Coffee Plants

The present invention also relates to kits comprising material that is of use for carrying out a method according to the invention. In particular, the present invention relates to kits for clonal and/or varietal identification of coffee plants, containing material for determining the insertion profile of Clem2 (or of another active LTR retrotransposon, in particular of an active LTR retrotransposon of the Clem2 family) in the genome of coffee plants. One of the advantages of the kits of the invention is that they can be used throughout the world (i.e., whatever the origin of the coffee plant or coffee tested) and for a large number of coffee plant species and/or varieties and clones.

In general, a kit according to the invention comprises at least one pair of primers for the amplification of specific regions of the coffee plant genomic DNA. A kit according to the invention can be designed so as to be used with a particular technique for determining the retrotransposon insertion profile, in particular an SSAP, REMAP or IRAP technique.

Thus, in certain embodiments, a kit according to the invention is designed so as to be used with the IRAP technique, and comprises at least one pair of primers for the amplification of coffee plant genomic DNA regions included between the LTRs of two close copies of Clem2 (or of another active LTR retrotransposon). In such embodiments, the pair of primers is made up of two primers according to the invention, as described above.

In other embodiments, a kit according to the invention is designed so as to be used with the REMAP technique, and comprises at least one pair of primers for the amplification of coffee plant genomic DNA regions included between an LTR of Clem2 (or of another active LTR retrotransposon) and a microsatellite. In such embodiments, the pair of primers consists of a primer according to the invention and of a primer of SSR type. Several SSR primers (of different sequences) can be included in the kit. SSR primers are known to those skilled in the art. They generally consist of from 15 to 20 nucleotides and comprise a microsatellite motif of two bases repeated 6 to 8 times (or a microsatellite motif of three bases repeated 4 or 5 times) and a few (for example one or two) random bases located 5′ or 3′ of the repeat motif.

In yet other embodiments, a kit according to the invention is designed so as to be used with the SSAP technique, and comprises at least one pair of primers for the amplification of coffee plant genomic DNA regions included between an LTR of Clem2 (or of another active LTR retrotransposon) and a restriction site. In such embodiments, the pair of primers consists of a primer according to the invention and of a primer complementary to the linker which is bonded by ligation to the ends of the fragments obtained by enzymatic digestion of the genomic DNA. As indicated above, the linkers are known in the art.

Depending on the technique for which it is designed, a kit may also comprise reagents or solutions for genomic DNA extraction, restriction enzymes, reagents or solutions for PCR amplification, reagents or solutions for separating amplicons according to their size, sequencing reagents or solutions, and/or detection means. Protocols for using these reagents and/or solutions can also be included in the kit.

The various components of the kit can be provided in solid form (for example in lyophilized form) or in liquid form. A kit can optionally comprise a container containing each of the reagents or solutions, and/or containers for carrying out certain steps of the method of the invention.

A kit according to the invention may also comprise instructions for carrying out a method of the invention in order to establish an insertion profile of Clem2 (or of another active LTR retrotransposon) in the coffee plant genome. The instructions for carrying out a method according to the invention can comprise instructions for extracting genomic DNA from coffee plant samples, instructions regarding enzymatic digestion and the ligation of linkers in the case of a kit intended to be used with the SSAP technique, instructions regarding the PCR amplification conditions, instructions regarding the separation of the amplicons obtained, and/or instructions for interpreting the results.

A kit according to the invention can also comprise instructions in the form recommended by a governmental agency regulating the preparation, sale and use of biological products.

Unless they are otherwise defined, all the technical and scientific terms used here have the same meanings as those commonly understood by an ordinary specialist in the field to which this invention belongs. All the publications and patent applications, all the patents and any other references mentioned herein are incorporated by way of reference.

### EXAMPLES

The following examples describe certain embodiments of the present invention. However, it is understood that the examples are given merely by way of illustration only, and do not in any way limit the scope of the invention.

### Materials and Methods

### Plant Material.

Four species of the Coffea genus were used in the present study: C. canephora and C. arabica originating from Africa and C. perrieri and C. mauritianae originating from Madagascar. Individuals resulting from three different genotypes, BA53 (originating from Ivory Coast, Guinean group), BD55 (originating from Cameroon, Congolese group) and HD200 (obtained from a natural haploid, genotype undergoing sequencing) for C. canephora, and the cultivated genotype “bourbon pointu” for C. arabica were used. All the individuals used in the present study are part of a live collection in the greenhouses of the IRD (Research Institute for Development) in Montpellier, France.

### Nucleic Acid and Protein Extraction and Quantification.

The genomic DNA extractions were carried out using 0.1 g of fresh young coffee plant leaves according to the protocol of the DNeasy Plant Mini kit from Qiagen. The total RNA extractions were carried out using fresh young leaves according to the protocol of the RNeasy Plant Mini kit from Qiagen, with the exception of C. arabica (bourbon pointu) RNAs derived from embryos and from albumen (which were kindly supplied by T. Joet of the IRD Montpellier). All the samples were treated with RNase-free DNase I according to the recommendations of the RNeasy Plant Mini kit from Qiagen or with RNase-free DNase I from Fermentas. The nucleic acids were assayed by spectrophotometry (NanoDrop 1000 Spectrophotometer), and the quality was estimated by agarose electrophoresis (1×TBE). Protein extractions were carried out using young coffee plant leaves according to the “Total Extraction Protein” protocol. The proteins were degraded in order to release the compounds present in these proteins using the total DNA extraction Mini preparation protocol.
Assembly and Identification in Silico of the Retrotransposons in C. canephora.
The genomic resources available to the team of the inventors: approximately 250 000 EST (Expressed Sequence Tag) sequences (200 to 800 bp), 6 C. canephora BAC clones, 10 C. arabica BAC clones and more than 750 genomic sequences (120 to 1200 bp) were used to carry out an assembly and to obtain more extended sequences, called “contigs”. The AAARF automated program (De Barry et al., BMC Bioinformatics, 2008, 9: 235) was used on the calculation server of the IRD Montpellier to carry out this assembly, according to standard parameters. The contigs obtained were used for a homology search (BLASTX) against an annotated bank of transposable element proteins (accessible on repbase www.girinst.org/repbase/). The large contigs exhibiting strong analogy with LTR retrotransposon proteins were analyzed manually and annotated using the Artemis software.

### Selection of Primers and PCR Amplification.

The primers used for all the PCR amplifications were designed in the noncoding (LTR) or coding regions by means of the annotations of the retrotransposons on Artemis with the Primers3 software (frodo.wi.mit.edu/). Unless otherwise indicated, the PCR conditions used were generally the following: 94° C., 2 minutes; 30 cycles of 94° C., 30 seconds; 55° C., 30 seconds; 72° C., 90 seconds and 5 minutes of final extension at 72° C. The migration was carried out at 100 V generally with a migration gel composed of 1% agarose, 1×TBE. The bands were visualized under UV after staining with ethidium bromide (ETB) for 15 minutes. The PCR amplifications on the genomic DNA (gDNA) were carried out with 25 ng of template DNA with a final volume of 20 μl according to the recommendations for the GoTaq enzyme from Promega for the composition of the reaction mixture. Identical PCR conditions were used for the amplifications of cDNA (prepared by reaction synthesis from total RNA or already available—Mahesh et al., Plant Cell Rep., 2006, 25: 986-992) and of RNA, with the exception of the number of cycles (40) and of the amount of template (1 μl of cDNA and 500 ng of total RNA).
Synthesis of cDNAs in C. canephora.
cDNA synthesis was carried out according to the protocol of the SuperScript III First Strand Synthesis System for RT-PCR kit from Invitrogen. One microgram of total RNA was used for each reaction with an Oligo(dT)₂₀ as antisense primer.

### Purification of the PCR Amplification Products and Sequencing.

The PCR products were extracted from the agarose gel and purified using the QIAquick PCR Purification kit (Qiagen). The amount of PCR products purified was assayed by spectrophotometry (Nanodrop). The sequencing reactions were carried out by an external company using one of the primers that had been used for the PCR amplification.

### Retrotransposon Microsatellite Amplified Polymorphism (REMAP).

Five ISSR primers among those defined by Joshi et al. (Theor. Appl. Genet., 2000, 100: 1311-1320) were used in combination with the REMAP-clem2-5′ primer (located at the 5′ end of the LTR upstream of the element). For each pair of primers (ISSR and REMAP/LTR-clem2), the amplification was carried out in a final volume of 15 μl with 25 ng of genomic DNA, 0.2 μM of the 2 primers, 0.2 mM of dNTPs, and 0.5 U of Promega GoTaq DNA Polymerase. PCR was carried out under the following conditions: 94° C., 2 minutes; 35 cycles of 94° C., 1 minute; 55° C., 1 minute, 72° C., 1 minute 30 seconds, and 8 minutes at 72° C.

### Example 1

### Identification of LTR Retrotransposons in the Coffee Plant

Using the C. canephora genomic resources that they had at their disposal, the inventors assembled highly repeated sequences (Assisted Automated Assembler of Repeat Families algorithm, AARF) and identified long sequences characteristic of the transposable elements of LTR retrotransposon (RTN) type. Using the assembled sequences (contigs), 29 different RTNs were identified, including 18 of Ty3-gypsy type, 10 of Ty1-copia type and 1 not characterized. Two, among these 29 RTNs, (Divo and Nana) had already been identified and described by the inventors (Hamon et al., Mol. Genet. Genomics, June 2011, 285: 447-460).

The 11 additional complete RTNs identified (i.e. Clem1.1, Clem4, Rom1, Clem2, Clem3.2, Clem5, Clem6, Clem11, Rom6, Rom10 and Rom13) have a size ranging from 13458 by for the largest (Clem2, Ty3-gypsy) to 4822 by for the smallest (Clem3.2, Ty1-copia). The LTRs of these RTNs also exhibit a great size variation ranging from 3042 by for Clem2 to 149 by for Clem3.2.

### Example 2

### Activity of the LTR Retrotransposons in Various Coffea Tissues

One of the objectives of this study was to estimate the expression of the LTR-RTNs reassembled in various tissues of C. canephora. Primers were designed for 21 RTNs in noncoding regions, the LTRs, but also in coding regions, the GAG (Group-specific Antigen), RT (Reverse Transcriptase), RH (RNase H) and IN (Integrase) protein domains. The primers were tested via PCR amplifications on the genomic DNA of the BA53-type individual of C. canephora originating from West Africa, Guineans genetic diversity group (Gomez et al., BMC Evol. Biol., 2009, 9: 167). The expression of the LTR-RTNs was then investigated by PCR amplification using, as template, cDNA libraries of leaves or of fruits of C. canephora coffee plants at various stages in maturation (Mahesh et al., Plant Cell Rep., 2006, 25: 986-992).

In order to complete these results, the expression of certain LTR-RTNs was analyzed by RT-PCR on other organs and tissues of C. arabica, such as the embryo and the albumen. A sequence homology search (BLAST) analysis of the LTR-RTNs was also carried out on public EST data (translated sequences) in C. canephora and C. arabica.

The results of the amplifications on the leaf and fruit cDNAs showed differences for the 21 LTR-RTNs used. Eleven LTR-RTNs (Divo, Nana, Rom1, Rom6, Rom10, Rom12, Rom14, Rom15, Rom16, Rom17, and Rom21) showed no amplification. Six LTR-RTNs (Clem2, Clem3.2, Clem4, Clem5, Rom4 and Rom13) were always amplified, irrespective of the region targeted and the origin of the cDNAs (leaves or fruits). Four LTR-RTNs (Clem1.1, Rom5, Rom8 and Clem11) showed variations in the amplifications according to the tissues and/or regions used.

The homology search in the EST banks identified three groups of LTR-RTNs: (1) those with no homology in C. canephora (3 LTR-RTNs—Divo, Nana, Rom10); (2) those with a limited number (<20) of ESTs (13 LTR-RTNs—Clem1.1, Clem3.2, Clem4, Clem5, Rom4, Rom6, Rom8, Clem11, Rom14, Rom15, Rom16, Rom17 and Rom21); and (3) those with a very large number (>20) of ESTs (5 LTR-RTNs—Clem2, Rom1, Rom5, Rom12 and Rom13).

Two LTR-RTNs, Clem2 and Clem4, respectively of Ty3-gypsy and Ty1-copia type, were selected for expression studies by RT-PCR in various tissues and various genotypes. Both showed amplifications in the leaf and fruit cDNAs, but Clem2 was found to exhibit a very large number of homologies in the EST sequences of C. canephora (75), unlike Clem4 (which exhibited only 2).

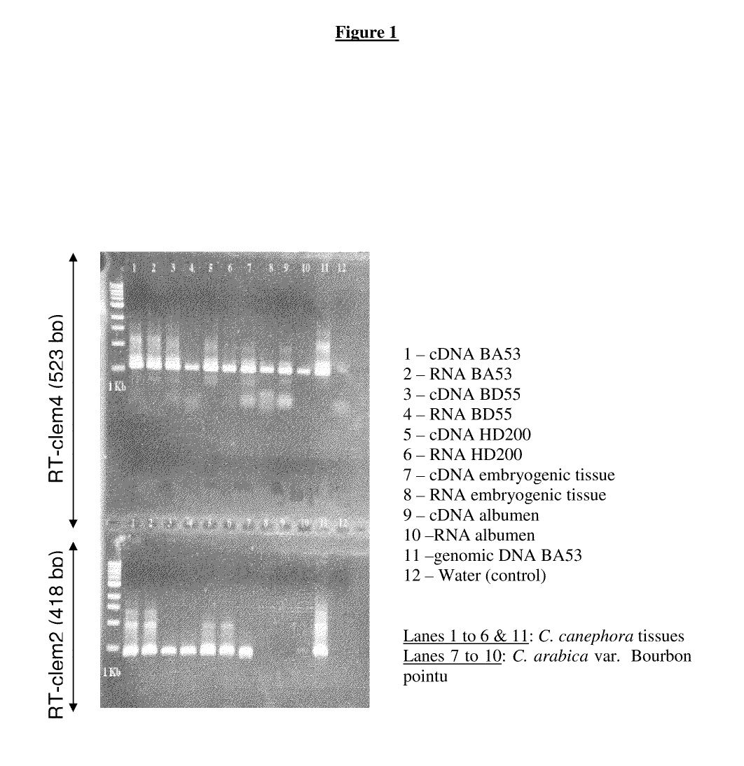

RNA extractions from leaves of coffee plants of genotypes BA53, BD55 and HD200 in C. canephora were carried out, and total RNAs from C. arabica bourbon pointu embryo and albumen were used to carry out RT-PCRs with the Clem2 and Clem4 “RT” primers (FIG. 1). The total RNA, pretreated with DNase I endonuclease, was used as a negative control for amplification and the genomic DNA was used as a positive control for amplification. The results obtained indicated amplifications for each of the templates used for RT-Clem4 (gDNA, cDNA and RNA) and also for RT-Clem2, with the exception of the embryonic tissue RNAs and the albumen cDNAs.

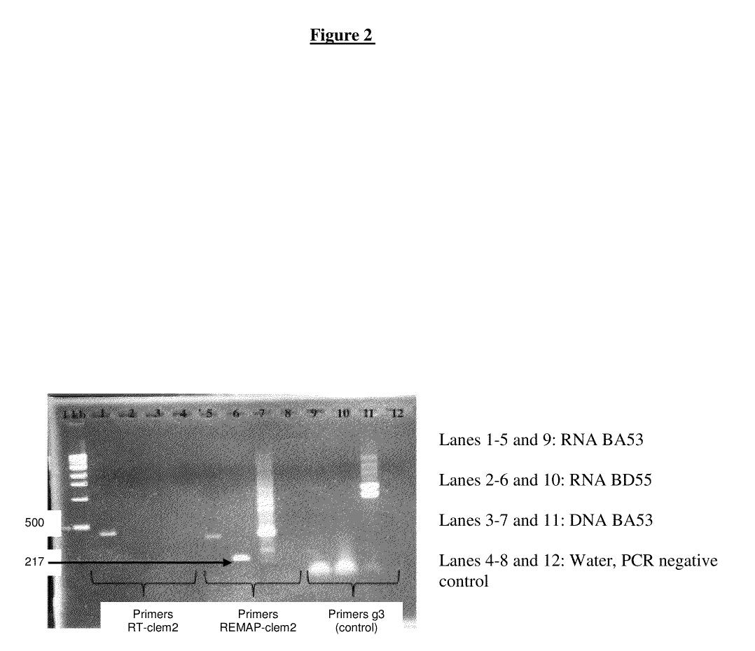

As shown in FIG. 2, PCR amplification with primers designed in the exons of the g3 gene (EIN4 locus, Guyot et al., BMC Plant Biol., 2009, 9: 22) was found to be positive for the gDNA of the BA53 genotype, whereas no amplification was observed on the total RNA for BA53 and BD55.

These results indicate that gDNA segments of Clem2 but not of the g3 gene are present in the total RNA extracts of the BA53 genotype, despite treatment of the samples with DNase I. These results suggest the presence of DNA fragments bound to one or more proteins which protect them against the action of an endonuclease such as DNase I. This association is observed in the case of the presence of circular replicative forms in a virus-like particle or linear forms before integration into the genome.

### Example 3

### Confirmation of Circular Replicative Forms of the Clem2 RTN

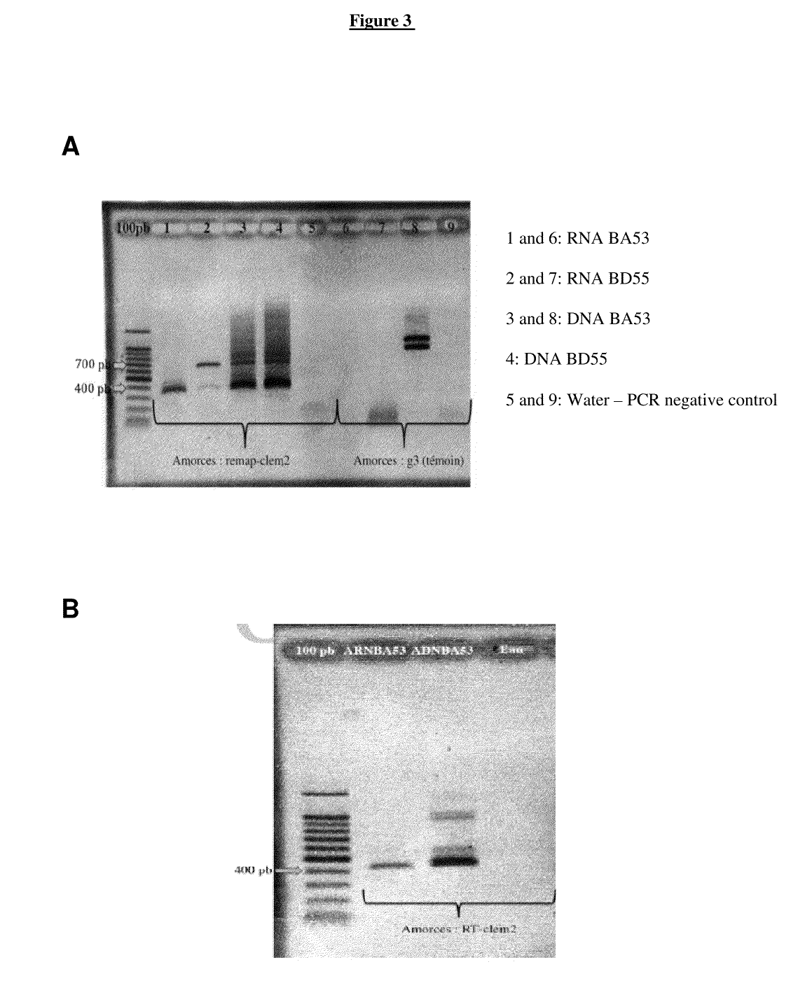

In order to confirm the presence of circular replicative forms for the Clem2 RTN, two primers (REMAP-clem2-5′ and REMAP-clem2-3′) were designed at the ends of the LTR regions of Clem2 and oriented towards the exterior of the RTN, and PCR amplifications were carried out on the genomic DNA and the total RNAs in C. canephora. The results showed amplifications on the total RNAs, of two different sizes (approximately 200 by and 400 bp), suggesting the presence of circular forms of DNA of the Clem2 LTR-RTN in the samples. FIG. 3(A) indeed confirms the presence of these bands after amplification with the remap-type pairs of primers on the BA53 and BD55 genotypes, and also after amplification on the RNAs of the BA53 genotype with the RT-clem2 pair of primers (FIG. 3(B)). The PCR amplification product for the REMAP-clem2 primers was purified and sequenced. Analysis of the sequence obtained for the 400 by band with remap-clem2 on BA53 showed that said sequence is homologous to the Clem2 LTRs. An amplification was also obtained using the RT-clem2 pair of primers, said amplification having a size of more than 400 bp, the expected size. This amplification was sequenced, but it was not possible to analyze, certainly due to the presence of a contamination.

Analogous amplifications for the Clem5, Rom13 and Clem4 elements were carried out on total RNAs in order to determine whether these replicative forms concerned only the Clem2 LTR-RTN. No amplification was observed on the BA53, BD55 and HD200 RNAs for Clem5 and Rom13, but amplifications were observed for Clem4 on the BA53, BD55 and HD200 RNAs.

These results confirm the presence of circular replicative forms for the Clem2 retrotransposon in the total RNA extracts in C. canephora.

### Example 4

### Analysis of the Insertion Polymorphism

In order to identify the Clem2 retrotransposon insertion polymorphism, a REMAP-type analysis was carried out on the two genotypes BA53 and BD55 of C. canephora. For each pair of primers used, one of the primers is specific for the 5′ LTR of Clem2 and is oriented towards the exterior of the retrotransposon, and the other primer is chosen from four different ISSR primers. After migration on a 2% agarose gel, the profiles obtained were different for the two genotypes, but identical for one and the same genome, whatever the ISSR primer used.

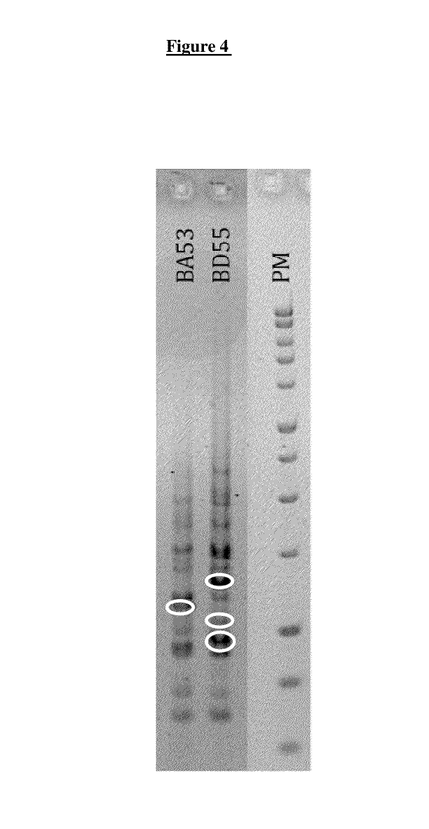

FIG. 4 shows the results of an TRAP analysis carried out on two genotypes of C. canephora (BA53 and BD55) with a single primer complementary to the end of the 5′ LTR of Clem2. The amplifications therefore here concern only the identical elements oriented head-to-tail. This analysis was carried out by agarose gel electrophoresis. This type of gel, which is easy to prepare, is not however highly resolving, but is already sufficient here to demonstrate differences between the two genotypes. The same amplification analyzed on an acrylamide gel would have made it possible to visualize differences of about one base (denaturing conditions).

### Example 5

### Estimation of the Clem2 and Clem5 Copy Number

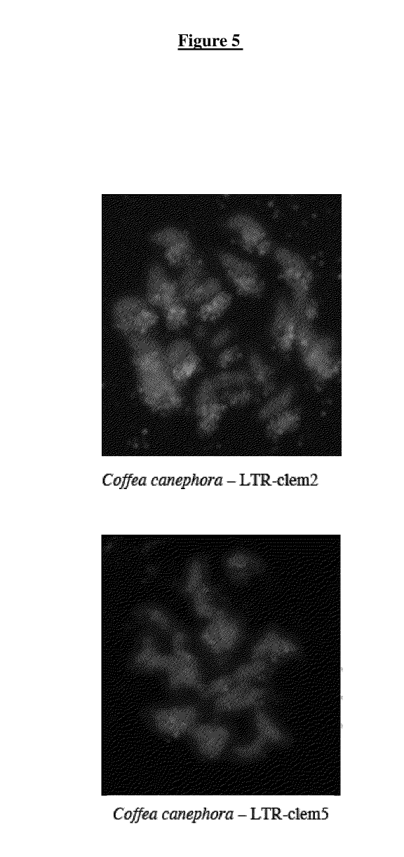

Two approaches were used to estimate the Clem2 and Clem5 RTN copy number in the C. canephora genome. Fluorescent in-situ hybridization (FISH) analyses for Clem2 and Clem5, and a hybridization on a “high-density” filter of a C. canephora (genotype HD200) BAC library for Clem2 were undertaken. The in situ hybridizations carried out on C. canephora metaphase chromosomes showed that Clem2 is present at a high copy number, whereas Clem5 is hardly present at all (FIG. 5).

Hybridization of the C. canephora BAC library using two probes specific for Clem2, one specific for the LTRs and the other specific for GAG, made it possible to obtain an estimation of the copy number for this element in the genome of this genotype. Under high stringency conditions, such as those used, a minimum number of 128 copies of Clem2 is found in the HD200 genome, genotype of the C. canephora species.

1-16. (canceled)

17. An isolated active LTR retrotransposon of the Clem2 family, wherein said retrotransposon has a nucleotide sequence comprising at each of its 5′ and 3′ ends, the sequence set forth in SEQ ID NO: 2 or a sequence consisting of 3042 nucleotides and having at least 80% homology with SEQ ID NO: 2.

18. The isolated active LTR retrotranspostion of the Clem2 family according to claim 17, wherein the sequence consisting of 3042 nucleotides has at least 85% homology with SEQ ID NO: 2.

19. The isolated active LTR retrotranspostion of the Clem2 family according to claim 17, wherein the sequence consisting of 3042 nucleotides has at least 90% homology with SEQ ID NO: 2.

20. The isolated active LTR retrotranspostion of the Clem2 family according to claim 17, wherein said retrotransposon hybridizes to SEQ ID NO: 1, or to the sequence complementary to SEQ ID NO: 1, under stringent hybridization conditions.

21. The isolated active LTR retrotranspostion of the Clem2 family according to claim 17, wherein said retrotranspostion is isolated from a coffee plant and/or is identical to a retrotransposon present in the genome of a coffee plant.

22. A primer comprising an oligonucleotide containing between 15 and 35 consecutive nucleotides of
(a) the nucleotide sequence set forth in SEQ ID NO: 2, or
(b) the sequence complementary to SEQ ID NO: 2, or
(c) a nucleotide sequence consisting of 3042 nucleotides and having at least 80% homology with the sequence set forth in SEQ ID NO: 2, or
(d) the sequence complementary to a nucleotide sequence consisting of 3042 nucleotides and having at least 80% homology with the sequence set forth in SEQ ID NO: 2.

23. The primer according to claim 22, wherein the sequence consisting of 3042 nucleotides has at least 85% homology with SEQ ID NO: 2.

24. The primer according to claim 22, wherein the sequence consisting of 3042 nucleotides has at least 90% homology with SEQ ID NO: 2.

25. The primer according to claim 22, wherein the oligonucleotide has the sequence set forth in SEQ ID NO: 3 or SEQ ID NO: 4.

26. The primer according to claim 22, further comprising a detectable label and/or a sequence of 1, 2, 3 or 4 random nucleotides.

27. A method for varietal and/or clonal identification of a coffee plant, comprising a step of: establishing the insertion profile, in the genome of the coffee plant tested, of an LTR retrotransposon of the Clem2 family as defined in claim 17.

28. The method according to claim 27, wherein establishing the insertion profile, in the genome of the coffee plant tested, of an LTR retrotransposon of the Clem2 family comprises determining insertion sites of the retrotransposon in the genome of the coffee plant.

29. The method according to claim 27, wherein the insertion profile is established using the SSAP (Sequence-Specific Amplification Polymorphism) technique, the REMAP (Retrotransposon-Microsatellite Amplified Polymorphism) technique, the IRAP (Inter-Retrotransposon Amplified Polymorphism) technique, the RBIP (Retrotransposon-Based Insertion Polymorphism) technique, or any variation thereof.

30. The method according to claim 27, wherein the method is carried out on total DNA extracted from a sample of the coffee plant tested, wherein the sample of the coffee plant is a sample of protoplast, organ, callus, seed, flower, fruit, leaf, stem, root, cutting or bean of the coffee plant tested.

31. The method according to claim 30, wherein the method is carried out on green coffee bean or a roasted coffee bean.

32. The method according to claim 27, wherein the step of establishing the insertion profile of the retrotransposon comprises amplifying the total DNA extracted from a sample of the coffee plant tested using at least one primer according to claim 4 to generate fragments specific of the insertion of the retrotransposon in the genome of the coffee plant tested.

33. The method according to 27 further comprising a step of: comparing the insertion profile obtained for the coffee plant tested with the insertion profile of the same retrotransposon in the genome of a control coffee plant of the same variety or of the same clone as the coffee plant tested.

34. The method according to claim 33, wherein the coffee plant tested and the control coffee plant both belong to the C. canephora species or to the C. arabica species.

35. The method according to claim 27, wherein the method is used for coffee certification and/or traceability.

36. A kit for varietal and/or clonal identification of coffee plants, comprising at least one primer according to claim 22.
